(19) United States
(12) Patent Application Publication
(10) Pub. No.: US 2024/0239814 A1
(43) Pub. Date: Jul. 18, 2024

(54) Complexes Of CU(I) As Antitumor Agents

(71) Applicant: UNIVERSITA´ DEGLISTUDI DI PADOVA, Padova (IT)

(72) Inventors: Valentina Gandin, Tarzo (IT); Cristina Marzano, Padova (IT); Marina Porchia, Padova (IT); Francesco Tisato, Padova (IT)

(21) Appl. No.: 18/569,713

(22) PCT Filed: Jun. 15, 2022
(86) PCT No.: PCT/EP2022/066390

§ 371 Date: Dec. 13, 2023

(30) Foreign Application Priority Data
Jun. 15, 2021 (IT) 102021000015635

(51) Int. Cl. C07F 1/00; A61P 35/00

(52) U.S. Cl. CPC C07F 1/005; A61P 35/00

(57) ABSTRACT

The invention relates to a Cu(1) complex of Formula (I), wherein L is a ligand of Formula (II), wherein n1, n2, n3 are independently to each other, an integer from 0 to 1, A1, A2 and A3 are independently from each other,—a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or—an heterocyclic ring selected from piperazinyl, morpholynyl, thiomorpholynyl, optionally substituted with (C₁-C₃)alkyl, where the heterocyclic ring is linked with the nitrogen atom to —CH₂— residue, with the proviso that when A1, A2 or A3 is optionally substituted phenyl, then n1, n2 or n3, respectively, is 0, and when A1, A2 or A3 is optionally substituted heterocyclic ring, then n1, n2 or n3, respectively, is 1, for use in the treatment of tumours.

### FIELD OF THE INVENTION

The present invention relates to complexes of Cu(I) as antitumor agents.

### BACKGROUND

The serendipitous discovery of the antitumor activity of cis-diamminedichloroplatinum(II) (cisplatin, CDDP) represents one of the most significant events for cancer chemotherapy in the 20th century (Ghosh S. Cisplatin: The first metal based anticancer drug. Bioorg Chem. 2019 July; 88:102925. 2. Jung, Y. W.; Lippard, S. J. Chem. Rev. 2007, 107, 1387).

CDDP and the second generation drugs cis-diammine-1-1′-cyclobutanedicarboxylatoplatinum(II) (carboplatin) and cyclohexane-1,2-diamineethanedioatoplatinum(II) (oxaliplatin, OXP) are highly effective in treating a variety of cancers, especially testicular cancer, for which the overall cure rate exceeds 90%.

However, the clinical use of platinum-based drugs is seriously limited by several side drawbacks, such as severe toxic effects on normal tissues, and by the early appearance of resistance phenomena. Actually, ongoing from the first generation drug CDDP to second generation of platinum-based compounds (carboplatin and OXP), the issue of reducing toxicity over normal cells and of widening the spectrum of action towards additional human cancers has been only partially addressed (Johnstone T C, Suntharalingam K, Lippard S J. The Next Generation of Platinum Drugs: Targeted Pt(II) Agents, Nanoparticle Delivery, and Pt(IV) Prodrugs. Chem Rev. 2016 Mar. 9; 116(5):3436-86).

Hence, with the aim of meeting this pharmaceutical challenge and of obtaining drugs endowed with a better pharmacological profile, huge efforts have been undertaken in the last four decades in order to develop innovative metal-based anticancer drugs. In this field, several research groups oriented their endeavors towards the development of metal-based molecules showing at their cores, elements other than platinum, with the idea that non platinum metal complexes should exert anticancer activity by mechanisms different from that of cisplatin, thus affording new potential therapeutic tools for the treatment of aggressive refractory neoplasia. Moreover, the possibility to use other antitumor-active metal-based complexes with improved pharmacological properties may help to find an answer for curing those tumours that express both inherited and acquired resistance to Pt(II) drugs (Simpson P V, Desai N M, Casari I, Massi M, Falasca M. Metal-based antitumor compounds: beyond cisplatin. Future Med Chem. 2019 January; 11(2):119-135).

Copper-based compounds have been originally investigated on the assumption that endogenous metals may be less toxic than non-essential metals for normal cells. Actually, copper is better tolerated than platinum due to the existence of natural biological pathways that regulate copper levels and detoxify the metal where necessary. Moreover, the altered metabolism of cancer cells and the different responses between normal and tumor cells to copper overload lay the basis for the identification of selective antineoplastic copper based drugs. In recent years, several classes of copper(I) and copper(II) complexes have been proposed as cytotoxic agents. At present, there is an increasing interest in developing copper complexes as anticancer agents. However, despite the great number of copper complexes developed so far, only one copper complex was very recently approved as a theranostic drug: the Cu(II)-dotatate derivative Detectnet®, produced by Curium Sas.

The coordination chemistry of copper is dominated by Cu(II) derivatives with few examples of Cu(I) compounds (Santini C, Pellei M, Gandin V, Porchia M, Tisato F, Marzano C. Advances in copper complexes as anticancer agents. Chem Rev. 2014 Jan. 8; 114(1):815-62).

The inventors principally focused their attention on copper(I) complexes because there is a general consensus among biochemists that physiological copper is primarily internalized in cells as Cu(I) rather than Cu(II) through the human copper transporter 1 (hCtr1). This transporter has been found overexpressed in many cancer cells, especially highly aggressive and metastatic cancer cells, and is required for tumourigenesis and cancer progression (Magistrato A, Pavlin M, Qasem Z, Ruthstein S. Copper trafficking in eukaryotic systems: current knowledge from experimental and computational efforts. Curr Opin Struct Biol. 2019 October; 58:26-33). On this basis, although copper(I/II) red-ox chemistry is an active machinery in living systems, the possibility to directly deliver copper(I) species to cell membrane may help cellular internalization of the metal, thereby enhancing its biological efficacy.

Phosphines (P) are very useful as copper(I) ligands, owing to their ability to stabilize copper in its reduced Cu(I) state, avoiding its disproportionation to Cu(0) and Cu(II). Moreover they can also allow a fine tuning of the hydro-lipophilic character of the resulting metal complexes.

The inventors developed several derivatives, either homoleptic (CuP4) and heteroleptic (CuP2X or CuP2X2 where X is a co-ligand) complexes, both charged and neutral ones.

Many of these complexes were endowed with a significant cytotoxic activity and among them the most promising derivative was the homoleptic complex

[Cu(thp)₄][PF₆] (thp=tris-hydroxymethylphosphine) (Brevetto italiano N. Brevetto 0001369596 dal titolo “Complessi idrossimetilfosfinici di rame e loro impiego come agenti antitumorali” e brevetto U.S. Pat. No. 9,114,149 dal titolo “[Cu(thp)₄]ₙ[X]-ₙ Compounds for the Treatment of a Broad Range of Human Solid Tumors, Including Refractory Tumors”).

This hydrophilic monocationic “CuP₄” type copper(I) complex is highly soluble and stable in water solution. [Cu(thp)₄][PF₆] (thp=tris(hydroxymethyl)phosphine) was tested against a very huge panel of human cancer cells deriving from many different histotypes in two-dimensional (2D) monolayer cell cultures and was compared with cisplatin (CDDP) and oxaliplatin (OXP). It was found to possess IC₅₀ values ranging from less than 0.3 μM to 3 μM, being up to 50- and 30-fold more cytotoxic than CDDP and OXP, respectively. In an additional panel of human cancer cell lines endowed with acquired or natural resistance to CDDP or to OXP, [Cu(thp)₄][PF₆] was found to be potent, with IC₅₀ values up to 35-fold lower than the reference metallodrugs. The calculation of the Resistant Factor (RF), which is defined as the ratio between IC₅₀ calculated for the resistant cells and those arising from the sensitive ones, indicated a striking lack of cross-resistance between [Cu(thp)₄][PF₆] and platinum drugs. Moreover, [Cu(thp)₄][PF₆] was markedly more potent in killing tumor cells than the reference agents in those tumour cells that, expressing high level of multidrug resistance (MDR) proteins, are cross resistant to various structurally and functionally unrelated anticancer drugs. (Marzano C., Gandin V., Pellei M., Colavito D., Papini G., Gioia Lobbia G., Del Giudice E., Porchia M., Tisato F., Santini C. (2008) In vitro antitumor activity of the water soluble copper(I) complexes bearing the tris(hydroxymethyl)phosphine ligand. J. Med. Chem., 51, 798-808. Gandin V., Pellei M., Tisato F., Porchia M., Santini C., Marzano C. (2012) A novel copper complex induces paraptosis in colon cancer cells via the activation of ER stress signalling. J. Cell. Mol. Med., 16, 142-51. Italian patent (MC2006A000059), “Complessi fined on May 16, 2006, N. 0001369596 granted on 18 Jan. 2010. Assignees: ASSOCIAZIONE DREAM MC, FONDAZIONE CASSA DI RISPARMIO DELLA PROVINCIA DI MACERATA. Inventors: GIOIA LOBBIA GIANCARLO (University di Camerino—MC), MARTINI DOMENICO (ACOM-Montecosaro—MC), SANTINI CARLO (Università di Camerino—MC), TISATO FRANCESCO (ICIS-CNR, Padova). U.S. Pat. No. (9,114,149), “[Cu(thp)4]n[X]-n Compounds for the Treatment of a Broad Range of Human Solid Tumors, Including Refractory Tumors”, Inventors: Cristina Marzano, Marina Porchia, Francesco Tisato, Valentina Gandin, Carlo Santini, Maura Pellei, Giancarlo Gioia Lobbia, Grazia Papini, filed by Università Degli Studi di Padova and Università degli Studi di Camerino, 16 Aug. 2011 (Priority to PCT/IB2011/053624); Assigned to Università Degli Studi di Padova and Università degli Studi di Camerino 6 Jun. 2014; 25 Aug. 2015 Application granted and Publication of U.S. Pat. No. 9,114,149B2).

[Cu(thp)⁴][PF⁶] was tested against three-dimensional (3D) cell cultures of human colon cancer cells, HCT-15 and LoVo, additionally, in both colon cancer spheroid models it showed IC₅₀ values very similar to those of OXP and about 2.5 lower than those obtained with CDDP (Gandin V., Ceresa C., Esposito G., Indraccolo S., Porchia M., Tisato F., Santini C., Pellei M., Marzano C. (2017) Therapeutic potential of the phosphino Cu(I) complex (HydroCuP) in the treatment of solid tumors. Sci Rep., 7, 13936)

As one of the most desired properties for an anticancer compound is to be selective toward cancer cells, it was also tested against human non transformed cells, and was found extremely selective towards cancer cells with respect to non-transformed cells, being the selectively index (defined as the quotient of the average IC₅₀ toward normal cells divided by the average IC₅₀ for the malignant cells) about 30 times better than that of CDDP and 3 times higher compared with that of OXP. From a mechanistic point of view, [Cu(thp)₄][PF₆] was shown to inhibit the proteolytic activities of proteasome, resulting in (unfolded) protein stress and, similarly to other copper complexes, to selectively kill cancer cells by triggering a programmed nonapoptotic pathway, namely paraptosis, that has been recently recognized as an important getaway to overcome apoptosis-resistance (Gandin V., Pellei M., Tisato F., Porchia M., Santini C., Marzano C. (2012) A novel copper complex induces paraptosis in colon cancer cells via the activation of ER stress signalling. J. Cell. Mol. Med., 16, 142-51).

The encouraging results obtained in in vitro studies, prompted us to test the therapeutic potential of [Cu(thp)₄][PF₆] in vivo, in syngenic and xenograft murine models of solid tumors. The toxicity profiles [Cu(thp)₄][PF₆] have been determined by means of acute toxicity studies in BALB/c and in C57BL mice. The median lethal doses (LD₅₀) and the maximum tolerated doses (MTD) calculated for [Cu(thp)₄][PF₆] were significantly higher that those recorded with CDDP (Gandin V., Ceresa C., Esposito G., Indraccolo S., Porchia M., Tisato F., Santini C., Pellei M., Marzano C. (2017) Therapeutic potential of the phosphino Cu(I) complex (HydroCuP) in the treatment of solid tumors. Sci Rep., 7, 13936; U.S. Pat. No. 9,114,149).

With respect to platinum drugs, [Cu(thp)₄][PF₆] induced a markedly higher reduction of tumor growth associated with minimal animal toxicity. Tested following different schedules in a highly aggressive syngeneic murine model, the Lewis Lung Carcinoma model, [Cu(thp)₄][PF₆] resulted in a dose-dependent inhibition of proliferation of tumor cell population in vivo which was superior to that induced by the gold-standard drug CDDP. Remarkably, animals treated with [Cu(thp)₄][PF₆] showed no clinical signs of toxicity and no anorexia, whereas those treated with CDDP appeared prostrate and showed substantial weight loss. Coherently, in human colorectal cancer xenografts, chemotherapy with [Cu(thp)₄][PF₆] was extremely effective in both oxaliplatin-sensitive and resistant models. The favorable in vivo tolerability of [Cu(thp)₄][PF₆] was also correlated to an encouraging biodistribution profile (Gandin V., Ceresa C., Esposito G., Indraccolo S., Porchia M., Tisato F., Santini C., Pellei M., Marzano C. (2017) Therapeutic potential of the phosphino Cu(I) complex (HydroCuP) in the treatment of solid tumors. Sci Rep., 7, 13936; U.S. Pat. No. 9,114,149).

An object of the present invention is hence to provide antitumor agents having at least an activity comparable to known complexes of Cu(I), having higher activity with respect to platinum drugs.

### SUMMARY OF THE INVENTION

In accordance with the present invention, the Applicant has surprisingly found out a new class of complexes of Cu(I), that were active as antitumor agents.

Specifically, the inventors noted that by using only two ligands, but each ligand having aromatic/heteroaromatic structure, the final complexes have antitumor activity even better than known antitumor agents as oxaliplatin or cisplatin.

In a first aspect therefore, the invention concerns a Cu(I) complex of Formula (I)

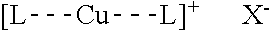

wherein L is a ligand of Formula (II)

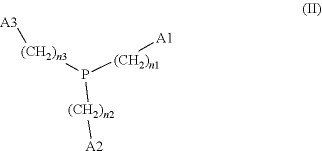

wherein
X⁻ is a monovalent anion,
n1, n2, n3 are independently to each other, an integer from 0 to 1,
A1, A2 and A3 are independently from each other,
   a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to —CH₂—
with the proviso that
when A1, A2 or A3 is optionally substituted phenyl, then n1, n2 or n3, respectively, is 0, and
when A1, A2 or A3 is optionally substituted heterocyclic ring, then n1, n2 or n3, respectively, is 1
for use in the treatment of tumours.

In second aspect the invention concerns a Cu(I) complex of Formula (I)

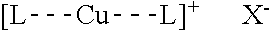

Wherein L is a ligand of Formula (II)

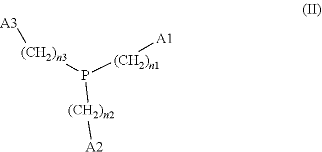

wherein
X⁻ is a monovalent anion
n1, n2, n3 are independently to each other, an integer from 0 to 1
A1, A2 and A3 are independently from each other,
   a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to —CH₂—
with the proviso that
when A1, A2 or A3 is optionally substituted phenyl, then n1, n2 or n3, respectively, is 0, and
when A1, A2 or A3 is optionally substituted heterocyclic ring, then n1, n2 or n3, respectively, is 1
for use as a medicament.

In another aspect, the invention concerns a Cu(I) complex of Formula (I)

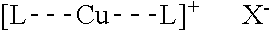

Wherein L is a ligand of Formula (II)

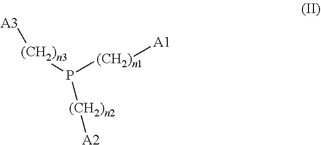

wherein
X⁻ is a monovalent anion
n1, n2, n3 are independently to each other, an integer from 0 to 1
A1, A2 and A3 are independently from each other,
   a phenyl optionally substituted with a substituent selected from the group consisting of carboxyl, sulphonyl hydroxyl, hydroxyl, and methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to —CH₂—
with the proviso that
when A1, A2 or A3 is optionally substituted phenyl, then n1, n2 or n3, respectively, is 0, and at least one of A1, A2 and A3 is a phenyl substituted with a substituent selected from the group consisting of carboxyl, sulphonyl hydroxyl, hydroxyl, and methoxy(C₁-C₃)alkoxy. and
when A1, A2 or A3 is optionally substituted heterocyclic ring, then n1, n2 or n3, respectively, is 1.

The Cu(I) complexes of the invention resulted to be very efficacious antitumor agents, and they were found to possess a cytotoxic activity even higher than cisplatin and oxaliplatin.

### BRIEF DESCRIPTION OF THE DRAWINGS

FIG. 1 represents body weight changes of example 3c.

### DETAILED DESCRIPTION OF THE INVENTION

Therefore the invention concerns a Cu(I) complex of Formula (I)

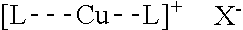

wherein L is a ligand of Formula (II)

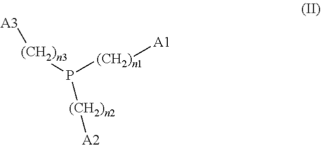

wherein
X⁻ is a monovalent anion
n1, n2, n3 are independently to each other, an integer from 0 to 1
A1, A2 and A3 are independently from each other,
   a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to —CH₂-,
with the proviso that
when A1, A2 or A3 is optionally substituted phenyl, then n1, n2 or n3, respectively, is 0, and
when A1, A2 or A3 is optionally substituted heterocyclic ring, then n1, n2 or n3, respectively, is 1
for use in the treatment of tumours.

The invention hence concerns complexes of Cu(I) of Formula (I) with two ligands L of Formula (II).

Each Ligand L comprises A1, A2, A3 equal or different from each other. A1, A2, A3 can be a phenyl optionally substituted with a substituent selected from the group consisting of (C₁-C₃)alkoxy, (C₁-C₃)alkyl, F, formyl, carboxyl, sulphonyl hydroxyl, hydroxyl, methoxy(C₁-C₃)alkoxy.

According to the invention, when A1, A2 or A3 is an optionally substituted phenyl, then n1, n2 or n3, respectively, is 0. Therefore when a phenyl is present it is directly linked to P atom.

Preferably at least one of A1, A2 and A3 is independently a phenyl substituted with a substituent selected from methyl, methoxy, F, formyl, sulphonyl hydroxyl, hydroxyl, metoxymetoxy and carboxyl.

In a more preferred embodiment the phenyl of A1, A2, A3, is substituted with methyl, F, or methoxy in para position.

In another preferred embodiment the phenyl of least one of A1, A2 and A3, is substituted with formyl, methoxy, sulphonyl hydroxyl, hydroxyl, methoxymethoxy or carboxyl in ortho position.

In a preferred embodiment A1, A2, A3 are independently from each other phenyl unsubstituted or substituted with methyl, methoxy or ethyl, more preferably in para position.

In another preferred embodiment one of A1, A2, A3 is phenyl substituted with carboxyl or sulphonyl hydroxyl or hydroxyl substituted in ortho position. A1, A2, A3 can be an heterocyclic ring selected from piperazinyl, morpholynyl, thiomorpholynyl, optionally substituted with (C₁-C₃)alkyl, where the heterocyclic ring is linked with the nitrogen atom to —CH₂—.

According to the invention, when A1, A2 or A3 is an optionally substituted heterocyclic ring, then n1, n2 or n3, respectively, is 1. According to the invention when the ligand contains a heterocyclic group, the latter is directly linked to CH₂ residue.

In a still another preferred embodiment A1, A2, A3 are independently from each other piperazinyl, more preferably substituted with methyl or ethyl.

In another preferred embodiment A1, A2, A3 are independently from each other morpholinyl or thiomorpholynyl, more preferably unsubstituted.

In a preferred embodiment A1, A2, A3 are equal.

In another preferred embodiment L comprises at least two of A1, A2, A3 equal. In this embodiment A1, A2, A3 are preferably optionally substituted phenyl.

In Formula (II) n1, n2 and n3 can be equal or different and they mean an integer from 0 to 1 dependently on the meaning of A1,A2 and A3, respectively.

In a preferred embodiment n1, n2, n3 are all 0, and A1, A2 and A3 are optionally substituted phenyl.

In another preferred embodiment n1, n2 and n3 are all 1, and A1, A2 and A3 are, independently from each other, optionally substituted pyridizinyl, optionally substituted morpholinyl or optionally substituted thiomorpholynyl.

X⁻ is a monovalent anion.

Preferably X⁻ is selected from the group consisting of BH₄⁻, NO₃⁻, PF₆⁻ and BF₄⁻.

In a still more preferred aspect the ligand L of Formula (II) is selected from the group consisting of:

| L | L |
|---|---|
| Formula (II) | Name |
| 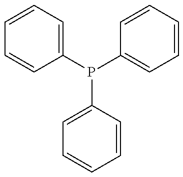 | PPh₃ 1 |
| 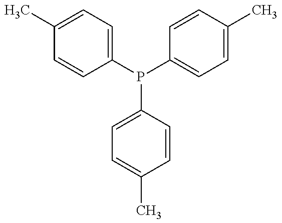 | P(p-tolyl)l₃ 2 |
| 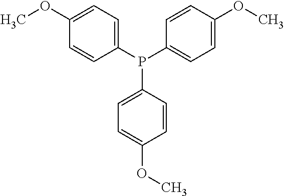 | P(p-OMe—C₆H₄)₃ 3 |
| 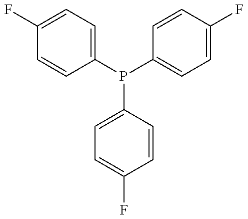 | P(p-F—C₆H₄)₃ 4 |
| 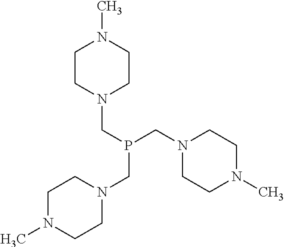 | PMepip 5 |
| 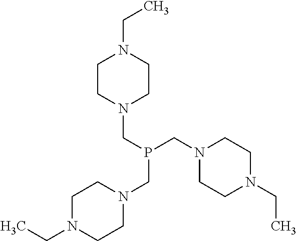 | PEtpip 6 |
| 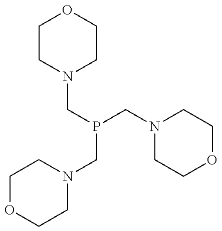 | Pmorf 7 |
| 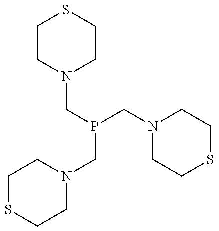 | Pthiomorf 8 |
| 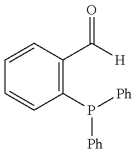 | DPBAL 9 |
| 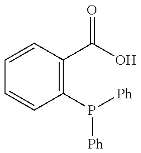 | DPBA 10 |
| 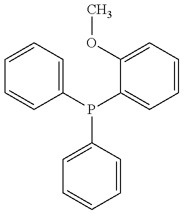 | DPMPP 11 |
| 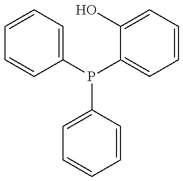 | POH 12 |
| 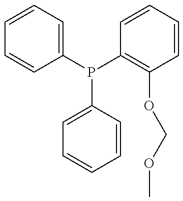 | TPPME 13 |
| 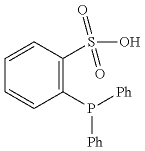 | TPPMS 14 |

The compound of Formula (I) is preferably selected from the group consisting of

| Compound | L | Formula (II) |
|---|---|---|
| CuL₂NO₃ | PPh₃ Bis[tris(phenylphosphine)] copper(I) nitrate 1 | 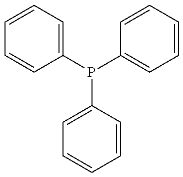 [SMILES: c1ccc(P(c2ccccc2)c2ccccc2)cc1] |
| CuL₂NO₃ | P(p-tolyl)l₃ Bis[tris(4- methylphenyl)phosphine] copper(I) nitrate 2 | 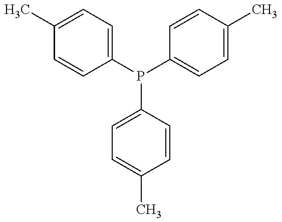 |
| CuL₂NO₃ | P(p-OMe—C₆H₄)₃ Bis[tris(4- methoxyphenyl)phosphine] copper(I) nitrate 3 | 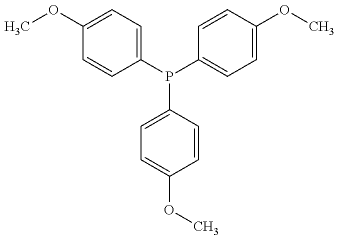 |
| CuL₂NO₃ | P(p-F—C₆H₄)₃ Bis[tris(4- fluorophenyl)phosphine] copper(I) nitrate 4 | 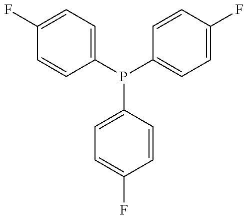 [SMILES: Fc1ccc(P(c2ccc(F)cc2)c2ccc(F)cc2)cc1] |
| CuL₂BF₄ | PMepip Bis[tris(N-methylpiperazineN′- methyl)phosphine]copper(I) tetrafluoroborate 5 | 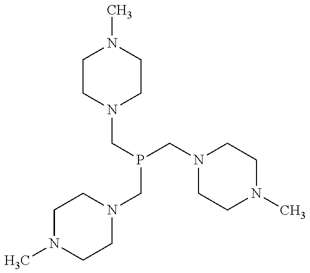 |
| CuL₂BF₄ | PEtpip Bis[tris(N-EthylpiperazineN′- methyl)phosphine]copper(I) tetrafluoroborate 6 | 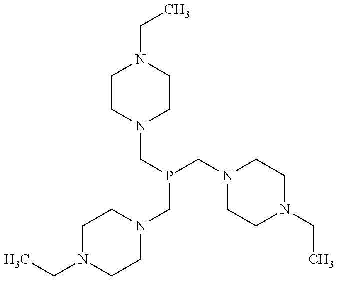 [SMILES: CCN1CCN(CP(CN2CCN(CC)CC2)CN2CCN(CC)CC2)CC1] |
| CuL₂BF₄ | Pmorf Bis[tris(morpholineN- methyl)phosphine]copper(I) tetrafluoroborate 7 | 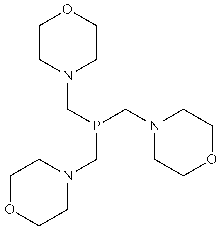 [SMILES: C1CN(CP(CN2CCOCC2)CN2CCOCC2)CCO1] |
| CuL₂BF₄ | Pthiomorf Bis[tris(thiomorpholineN- methyl)phosphine]copper(I) tetrafluoroborate 8 | 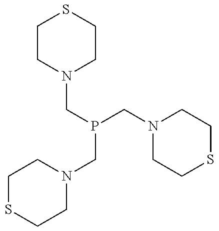 [SMILES: C1CN(CP(CN2CCSCC2)CN2CCSCC2)CCS1] |
| CuL₂BF₄ | DPBAL Bis[2-(diphenylphosphanyl) benzaldehyde]copper(I) tetrafluoroborate 9 | 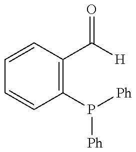 [SMILES: O=Cc1ccccc1P(c1ccccc1)c1ccccc1] |
| CuL₂BF₄ | DPBA Bis[2- (diphenylphosphanyl)benzoic acid]copper(I) tetrafluoroborate 10 | 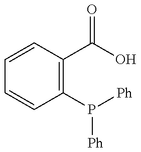 [SMILES: O=C(O)c1ccccc1P(c1ccccc1)c1ccccc1] |
| CuL₂BF₄ | DPMPP Bis[diphenyl(2- methoxyphenyl)phosphine] copper(I)tetrafluoroborate 11 | 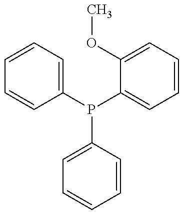 [SMILES: COc1ccccc1P(c1ccccc1)c1ccccc1] |
| CuL₂BF₄ | POH Bis[diphenyl(2- hydroxyphenyl)phosphine] copper(I)tetrafluoroborate 12 | 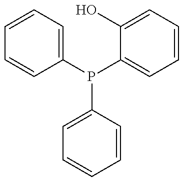 [SMILES: Oc1ccccc1P(c1ccccc1)c1ccccc1] |
| CuL₂BF₄ | TPPME Bis[diphenyl(2- (methoxymethoxy)phenyl) phosphine] copper(I)tetrafluoroborate 13 | 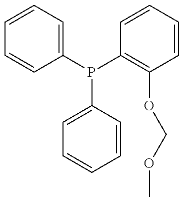 [SMILES: COCOc1ccccc1P(c1ccccc1)c1ccccc1] |
| CuL₂BF₄ | TPPMS Bis[2- (diphenylphosphino] benzenesulfonic acid]copper(I) tetrafluoroborate 14 | 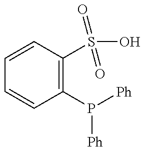 [SMILES: O=S(=O)(O)c1ccccc1P(c1ccccc1)c1ccccc1] |

The inventors surprisingly found out that the compounds of Cu(I) were antitumor agent.

Therefore, the invention concerns a Cu(I) complex of Formula (I)

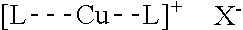

wherein L is a ligand of Formula (II)

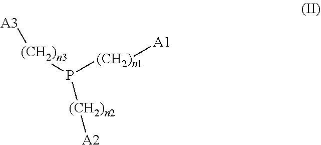

wherein
X⁻ is a monovalent anion
n1, n2, n3 are independently to each other, an integer from 0 to 1
A1, A2 and A3 are independently from each other,
   a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to —CH₂—
with the proviso that
when A1, A2 or A3 is optionally substituted phenyl, then n1, n2 or n3, respectively, is 0, and
when A1, A2 or A3 is optionally substituted heterocyclic ring, then n1, n2 or n3, respectively, is 1
for use as a medicament.

Preferred embodiments for A1, A2, A3 above indicated for the compounds for use in the treatment of tumours are here recalled for the Cu(I) complex of Formula (I) for use as a medicament.

In another aspect, the invention concerns a Cu(I) complex of Formula (I)

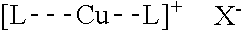

Wherein L is a ligand of Formula (II)

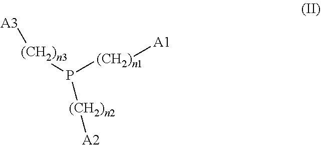

wherein
X⁻ is a monovalent anion
n1, n2, n3 are independently to each other, an integer from 0 to 1
A1, A2 and A3 are independently from each other,
   a phenyl optionally substituted with a substituent selected from the group consisting of carboxyl, sulphonyl hydroxyl, hydroxyl, and methoxy(C₁-C₃)alkoxy or
   an heterocyclic ring selected from piperazinyl, morpholynyl, thiomorpholynyl, optionally substituted with (C₁-C₃)alkyl, where the heterocyclic ring is linked with the nitrogen atom to the residue —CH₂—,
with the proviso that
when A1, A2 or A3 is an optionally substituted phenyl, then n1, n2 or n3, respectively, is 0, and at least one of A1, A2 and A3 is a phenyl substituted with a substituent selected from the group consisting of carboxyl, sulphonyl hydroxyl, hydroxyl, and methoxy(C₁-C₃)alkoxy. and
when A1, A2 or A3 is an optionally substituted heterocyclic ring, then n1, n2 or n3, respectively, is 1.

The invention hence concerns new complexes of Cu(l) of Formula (I) with two ligands L of Formula (II).

Each Legand L comprises A1, A2, A3, equal or different from each other. A1, A2, A3 can be a phenyl optionally substituted with sulphonyl hydroxyl, hydroxyl or carboxyl. When A1, A2 or A3 is optionally substituted phenyl, then n1, n2 or n3, respectively, is 0 and at least one of A1, A2 and A3 is a phenyl substituted with a substituent selected from the group consisting of carboxyl, sulphonyl hydroxyl, hydroxyl, and methoxy(C₁-C₃)alkoxy.

In a preferred embodiment at least one of A1, A2, A3 is, phenyl substituted with hydroxyl, sulphonyl hydroxyl, metoxymetoxy or carboxyl in ortho position.

A1, A2, A3 can be an heterocyclic ring selected from piperazinyl, morpholynyl, thiomorpholynyl, optionally substituted with (C₁-C₃)alkyl, where the heterocyclic ring is linked with the nitrogen atom to the residue —CH₂ —. According to the invention, when A1, A2 or A3 is optionally substituted heterocyclic ring, then n1, n2 or n3, respectively, is 1.

In another preferred embodiment one of A1, A2, A3 is phenyl substituted with carboxyl, methoxy, sulphonyl hydroxyl, methoxymethoxy or hydroxyl, more preferably substituted in ortho position.

In a still another preferred embodiment A1, A2, A3 are independently from each other piperazinyl, more preferably substituted with methyl or ethyl.

In another preferred embodiment A1, A2, A3 are independently from each other morpholinyl or thiomorpholynyl, more preferably unsubstituted.

In a preferred embodiment A1, A2, A3 are equal.

In another preferred embodiment L comprises at least two of A1, A2, A3 equal. In this embodiment A1, A2, A3 are preferably optionally substituted phenyl. In a still another preferred embodiment one of A1, A2, A3 is a substituted phenyl.

In a preferred embodiment n1, n2, n3 are all 0, and at least one of A1, A2 and A3 is a substituted phenyl.

In another preferred embodiment n1, n2 and n3 are all 1, and A1, A2 and A3 are, independently from each other, optionally substituted pyridizinyl, optionally substituted morpholinyl or optionally substituted thiomorpholynyl.

X⁻ is a monovalent anion.

Preferably X⁻ is selected from the group consisting of BH₄⁻, NO₃⁻, PF₆⁻ and BF₄⁻. In a still more preferred aspect the ligand L of Formula (II) is selected from the group consisting of:

| L | |
|---|---|
| Formula (II) | L |
| 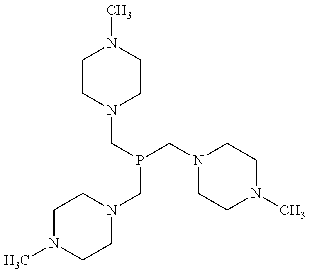 | PMepip 5 |
| 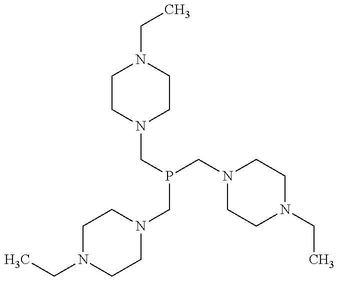 | PEtpip 6 |
| 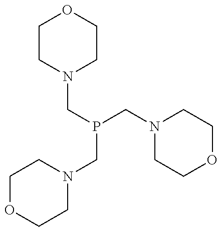 | Pmorf 7 |
| 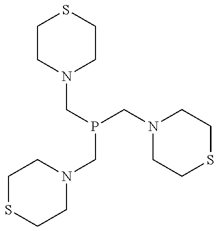 | Pthiomorf 8 |
| 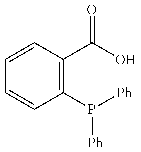 | DPBA 10 |
| 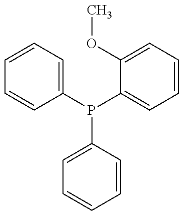 | DPMPP 11 |
| 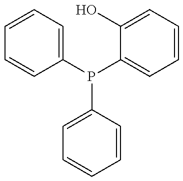 | POH 12 |
| 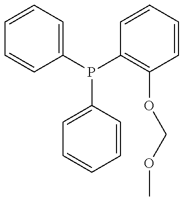 | TPPME 13 |
| 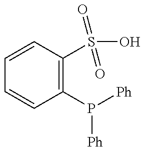 | TPPMS 14 |

The compound of Formula (I) is preferably selected from the group consisting of:

| Compound | L | Formula (II) |
|---|---|---|
| CuL₂BF₄ | PMepip 5 | 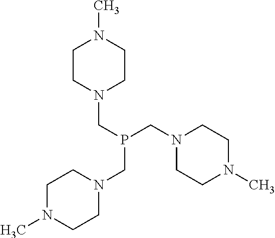 |
| CuL₂BF₄ | PEtpip 6 | 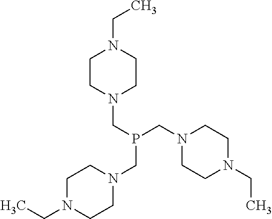 [SMILES: CCN1CCN(CP(CN2CCN(CC)CC2)CN2CCN(CC)CC2)CC1] |
| CuL₂BF₄ | Pmorf 7 | 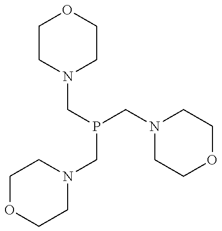 [SMILES: C1CN(CP(CN2CCOCC2)CN2CCOCC2)CCO1] |
| CuL₂BF₄ | Pthiomorf 8 | 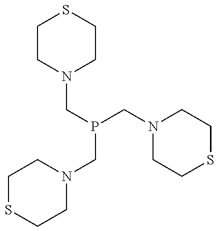 [SMILES: C1CN(CP(CN2CCSCC2)CN2CCSCC2)CCS1] |
| CuL₂BF₄ | DPBA 10 | 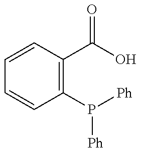 [SMILES: O=C(O)c1ccccc1P(c1ccccc1)c1ccccc1] |
| CuL₂BF₄ | DPMPP 11 | 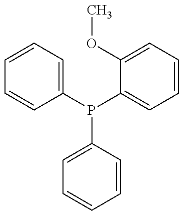 [SMILES: COc1ccccc1P(c1ccccc1)c1ccccc1] |
| CuL₂BF₄ | POH 12 | 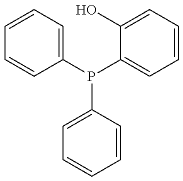 [SMILES: Oc1ccccc1P(c1ccccc1)c1ccccc1] |
| CuL₂BF₄ | TPPME 13 | 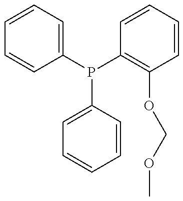 [SMILES: COCOc1ccccc1P(c1ccccc1)c1ccccc1] |
| CuL₂BF₄ | TPPMS 14 | 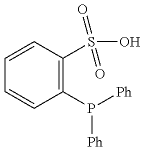 [SMILES: O=S(=O)(O)c1ccccc1P(c1ccccc1)c1ccccc1] |

The present invention is also directed to a pharmaceutical composition comprising Cu(I) complex of Formula (I) and a pharmaceutically acceptable carrier.

The invention relates also to a Cu(I) complex of Formula (I) as above indicated for use as a medicament.

The Cu(I) complexes of the invention may be administered by any suitable route of administration, including both systemic administration and topical administration.

Systemic administration includes oral administration, parenteral administration, trans-dermal administration, rectal administration, and administration by inhalation. The Cu(I) complexes of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that Cu(I) complex, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan.

The Cu(I) complexes of the invention will be normally, but not necessarily, formulated into a pharmaceutical composition prior to administration to a patient.

The pharmaceutical compositions of the invention are prepared using techniques and methods known to those skilled in the art.

The pharmaceutical compositions of the invention may be prepared and packaged in bulk form wherein an effective amount of a Cu(I) complex of the invention can be extracted and then given to the patient such as with powders, syrups, and solutions for injection. Alternatively, the pharmaceutical compositions of the invention may be prepared and packaged in unit dosage form. A dose of the pharmaceutical composition contains at least a therapeutically effective amount of the Cu(I) complex of this invention. When prepared in unit dosage form, the pharmaceutical compositions may contain from 1 mg to 1000 mg of the Cu(I) complex of this invention.

Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) trans-dermal administration such as trans-dermal patches; (4) rectal administration such as suppositories; (5) inhalation such as aerosols and solutions; and (6) topical administration such as creams, ointments, lotions, pastes, sprays and gels.

Suitable pharmaceutically acceptable excipients include the following types of excipients: diluents, fillers, binders, disintegrants, lubricants, granulating agents, coating agents, wetting agents, suspending agents, emulsifiers, sweeteners, flavour masking agents, colouring agents, anti-caking agents, humectants, plasticizers, viscosity increasing agents, antioxidants, preservatives, stabilizers, surfactants, and buffering agents. Suitable diluents and fillers include lactose, sucrose, dextrose, mannitol, sorbitol, starch, cellulose, calcium sulphate, and dibasic calcium phosphate. The oral solid dosage form may further comprise a binder. Suitable binders include starch, gelatine, sodium alginate, alginic acid, guar gum, povidone, and cellulose and its derivatives (e.g. microcrystalline cellulose). The oral solid dosage form may further comprise a disintegrant. Suitable disintegrants include crospovidone, sodium starch glycolate, alginic acid, and sodium carboxymethyl cellulose. The oral solid dosage form may further comprise a lubricant. Suitable lubricants include stearic acid, magnesium stearate, calcium stearate, and talc. Suitable carriers for oral dosage forms include but are not limited to magnesium carbonate, magnesium stearate, talc, lactose, pectin, dextrin, starch, methylcellulose, sodium carboxymethyl cellulose, and the like. Techniques used to prepare oral formulations are the conventional mixing, granulation and compression or capsules filling.

The compounds of the present invention may be also formulated for parenteral administration with suitable carriers including aqueous vehicles solutions (i.e.: saline, dextrose) or and/or oily emulsions.

In a still further aspect the invention relates to a Cu(I) complex of the invention for use in the treatment of a tumour.

Among the tumours the following can be cited:
Primary brain tumors including, but not limited to, astrocytic and oligodendroglia tumors such as astrocytomas, and glioblastomas;
Nerve tissue tumors of infancy such as neuroblastomas;
Metastatic brain tumors including, but not limited to, lung cancer, breast cancer, melanoma metastases, colon/colorectal metastases, kidney/renal metastases;
Solid tumors including, but not limited to, primary, recurrent or metastatic, progressive thyroid, breast, liver, bladder and kidney cancers.

Preferably the tumours are selected from the group consisting of cervical tumor, colon tumor, lung tumor, pancreatic tumor, ovarian tumor and breast tumor.

A therapeutically “effective amount” is intended to mean that amount of a compound that, when administered to a patient in need of such treatment, is sufficient to effect treatment, as defined herein. Thus, e.g., a therapeutically effective amount of a Cu(I) complex of the invention is a quantity of an inventive agent that, when administered to a human in need thereof, is sufficient so as to treat a tumour. The amount of a given compound that will correspond to such an amount will vary depending upon factors such as the particular compound (e.g., the potency (IC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the human in need of treatment (e.g., weight), disease or condition and its severity and the specific composition and method being used, but can nevertheless be determined by one of skill in the art.

### EXPERIMENTAL PART

### Example 1

### General Procedure for the Preparation of Compounds 1-15

Two different synthetic procedures were used for the synthesis of [CuP₂]X complexes

### i) With a Copper(I) Precursor (Compounds 4-14):

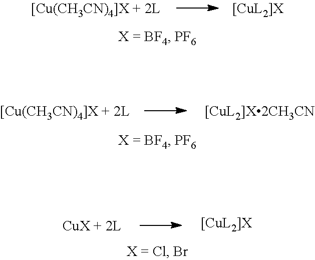

[CuL₂]BF₄ copper complexes 4-14 were prepared by an exchange reaction with the starting labile compound Cu(CH₃CN)₄BF₄ in acetonitrile using a stoichiometric amount of the pertinent legand L. The reaction mixture was kept at room temperature under nitrogen for 1-6 h, then the solvent was removed and the residue recovered and dried under vacuum overnight.

Specific work-up procedures were employed depending on the nature of L. In the case of L=PMepip, PEtpip, Pmorf, Pthiomorf, DPMPP, POH and 2-(Diphenylphosphino)(OCH₂OCH₃) after solvent removal a waxy white residue was obtained which was treated with diethylether. The products (compounds 5-8 and 11-13) as white solid were recovered by filtration and dried under vacuum.

Here, as an example, the synthesis of compound 5 was detailed:

To an acetonitrile solution (20 ml) of [Cu(CH₃CN)₄][BF₄] (100 mg, 0.32 mmol) PMepip (243 mg, 0.66 mmol) was added at room temperature under nitrogen. The colourless reaction mixture was stirred at room temperature for 1 h 30 min and then the solvent was removed under a gentle stream of dinitrogen leaving an oily residue.

The residue was treated with diethylether, filtered and the obtained white solid dried under vacuum for 2 days.

ii) Reduction of Nitrate Cu(II) Salt With Phosphine Oxide Formation, Followed in Case by Metathetical Reaction of Anion Exchange (Compounds 1-3) (ref. Gysling, H., & Kubas, G. (1979). “Coordination Complexes of Copper(I) Nitrate” Inorg. Synth. Vol. 19, pp. 92-97)

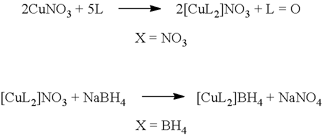

This procedure was used for the synthesis of compounds 1-3. The reaction took place in hot methanol without any precautions for the exclusion of air. The phosphine was dissolved in methanol under stirring and then the Cu(Il) salt was added in small portions. The salt dissolved immediately with the formation of a clear colorless solution, but near the end of the addition a heavy white precipitate forms. After addition of all the copper salt the reaction suspension was heated at reflux for about 10 minutes and then cooled to ambient temperature. The white precipitate was filtered and washed with ethanol and diethyl ether to eliminate the phosphinoxide formed as a side product. The Cu(I) derivative was dried under vacuum and recrystallized from ethanol or methanol.

### Example 2

### Characterization of [CuL₂]X Compounds

Compound CuL₂NO₃4 L=P(p-F—C₆H₄)₃

By following the procedure ii), compound Cu(P(p-F—C₆H₄)₃)₂ NO₃ was obtained as white solid.

MW: 758

Anal. Calcd. For CuP₂C₃₆F₆H₂₄NO₃: C 57.04, H 3.19, N 1.85%. Found: C 60.18, H 3.42, N 1.20%.

³¹P-NMR in CDCl₃: δ (ppm) −3.67 (s)

¹H-NMR in CDCl₃: δ (ppm) 7.04 (t, [Cu[P[C(CHCH)₂C—F]₃]₂][NO₃]); 7.22-7.30 (m, [Cu[P[C(CHCH)₂C—F]₃]₂][NO₃]).

Compound CuL₂ BF₄5 L=P(PMepip)₃

By following the procedure above reported compound Cu(P(PMepip)₃)₂ BF₄ was obtained as white solid.

MW: 891.4 White solid.

Anal. Calcd. for CuP₂N₁₂C₃₆H₇₈BF₄: C 48.51, H 8.82, N 18.86%

³¹P NMR (CDCl₃): δ(ppm) −30.42 (s)

¹H NMR (CDCl₃): δ(ppm) 2.29 (s, [Cu[P[CH₂N(CH₂CH₂)N—CH₃]₃]₂); 2.45 (s, [Cu[P[CH₂N(CH₂CH₂)N—CH₃]₃]₂][BF₄]); 2.66 (s, [Cu[P[CH₂N(CH₂CH₂)N—CH₃]₃]₂); 2.92 (s, [Cu[P[CH₂N(CH₂CH₂)N—CH₃]₃]₂).

ESI (+)-full-MS in MeOH (m/z assignment, intensity %): 803 ([Cu[P[CH₂N(CH₂CH₂)NCH₃]₃]₂]⁺, 100); 433 ([CuP[CH₂N(CH₂CH₂)NCH₃]₃]⁺, 45).

Compound CuL₂ BF₄ 6 L=P(PEtpip)₃

By following the procedure above reported compound Cu(P(PEtpip)₃)₂ BF₄ was obtained as white powder.

MW 975.5

Anal. Calcd. for CuP₂N₁₂C₄₂H₉₀BF₄ C 51.71, H 9.30, N 17.22%. Found: C 50.21, H 9.98, N 18.65%.

³¹P NMR (D₂O): δ (ppm) −35.13 (bs)

¹H NMR (D₂O): δ (ppm) 2.92 (bs, 2H, [Cu[P[CH₂N₂(CH₂CH₂)₂CH₂CH₃]₃]₂); 2.62 (dd, [Cu[P[CH₂N₂(CH₂CH₂)₂CH₂CH₃]₃]₂); 2.37 (q, [Cu[P[CH₂N₂(CH₂CH₂)₂CH₂CH₃]₃]₂); 0.96 (t, 3H, [Cu[P[CH₂N₂(CH₂CH₂)₂CH₂CH₃]₃]₂).

ESI-MS(+) in MeOH (m/z assignment, % intensity): 887 ([Cu[P(CH₂Pip-Et)₃]₂]⁺, 100); 475 ([CuP(CH₂Pip-Et)₃]⁺, 83); 511 ([Cu[P(CH₂Pip-Et)₃](H₂O)₂]⁺, 10).

Compound CuL₂ BF₄7 L=P(Pmorf)₃

By following the procedure above reported compound Cu(P(Pmorf)₃)₂ BF₄ was obtained as white powder.

MW 813

Anal. Calcd. for CuP₂N₆O₆C₃₀H₆₀BF₄ C 44.31, H 7.44, N 10.34%. Found: C 44.51, H 7.23, N 10.04%

³¹P NMR (D₂O): δ (ppm) −44.5 (bs)

¹H NMR (D₂O): δ (ppm) 3.68 (t, 4H, Cu[P[CH₂N(CH₂CH₂)₂O]₃]₂); 2.63 (s, 2H, Cu[P[CH₂N(CH₂CH₂)₂O]₃]₂]); 2.92 (bt, 4H, Cu[P[CH₂N(CH₂CH₂)₂O]₃]₂])

ESI-MS(+) in MeOH (m/z assignment, intensity %): 725 ([Cu[P(CH₂morph)₃]₂]⁺, 100); 332 ([P(CH₂morph)₃+H]⁺, 55); 394 ([CuP(CH₂morph)₃]⁺, 18)

Compound CuL₂ BF₄8 L=P(Pthiomorf)₃

By following the procedure above reported compound Cu(P(Pthiomorf)₃)₂ BF₄ was obtained as a beige solid.

MW: 909.5

Anal. Calcd. for CuP₂N₆S₆C₃₀H₆₀BF₄ C 39.62, H 6.65, N 9.24, S 21.15%.

³¹P NMR (DMSO): δ(ppm) −36.65 (bs)

¹H NMR (DMSO): δ(ppm) (s, 2.61 [Cu[P[CH₂N(CH₂CH₂)₂S]₃]₂][BF₄]); 2.75 (s, [Cu[P[CH₂N(CH₂CH₂)₂S]₃]₂][BF₄]); 2.80 (s, [Cu[P[CH₂N(CH₂CH₂)₂S]₃]₂][BF₄]).

Compound CuL₂ BF₄9 L=P(DPBAL)₃

By following the procedure above reported compound Cu(P(DPBAL)₃)₂ BF₄ was obtained as a yellow-orange solid.

MW: 731

Anal. Calcd. for CuP₂C₃₈O₂H₃₀BF₄: C 62.44, H 4.14% Found: C 58.22, H 4.11%

³¹P NMR (CDCl₃): δ(ppm) 1.95 (s).

¹H NMR (CDCl₃): δ(ppm) 9.83 (s, [Cu[(Cₐ—C_{b}—CHO(CH_{c}—CHₐ—CHₑ—CH_{f})—(C₆H₅)₂]₃]₂]); 7.65 (m, [Cu[(Cₐ—C_{b}—CHO(CH_{c}—CHₐ—CHₑ—CH_{f})—(C₆H₅)₂]₃]₂]; 7.42, 7.27 (m, [Cu[P[(Cₐ—C_{b}—CHO(CH_{c}—CHₐ—CHₑ—CH_{f})—(C₆H₅)₂]₃]₂]); 6.99 (m, [Cu[P[(Cₐ—C_{b}—CHO(CH_{c}—CHₐ—CHₑ—CH_{f})—(C₆H₅)₂]₃]₂]); 7.99 (m, [Cu[P[(Cₐ—C_{b}—CHO(CH_{c}—CHₐ—CHₑ—CH_{f})—(C₆H₅)₂]₃]₂])

¹³C NMR (CDCl₃): δ(ppm) 192.49 (s, [Cu[P[(Cₐ—C_{b}—CHO(CH_{c}—CHₐ—CHₑ—CHᵣ)—(C₆H₅)₂]₃]₂].

ESI (+)-full-MS in MeOH (m/z assignment, intensity %): 643 ([Cu[P[(Cₐ—C_{b}—CHO(CH_{c}—CHₐ—CHₑ—CH_{f})—(C₆H₅)₂]₃]₂]⁺, 100); 291 ([PH[(Cₐ—C_{b}—CHO(CH_{c}—CHₐ—CHₑ—CH_{f})—(C₆H₅)₂]₃]⁺, 51); 321 ([P(OCH₃) [(Cₐ—C_{b}—CHO(CH_{c}—CHₐ—CHₑ—CH_{f})—(C₆H₅)₂]₃]⁺, 42).

Compound CuL₂ BF₄ 10 L=P(DPBA)₃

By following the procedure above reported compound Cu(P(DPBA)₃)₂ BF₄ was obtained as a pale yellow solid.

MW 763

Anal. Calcd. for CuP₂C₃₈O₄H₃₀BF₄: C 59.82, H 3.96% Found: C 56.47, H 4.10%.

¹H NMR (CDCl₃): δ(ppm) 6.46 (s, [Cu[P[Cₐ—C_{b}—COOH(CH_{c}—CHₐ—CHₑ—CH_{f})—(C₆H₅)₂]₃]₂]); 6.96 (d, [Cu[P[Cₐ—C_{b}—COOH(CH_{c}—CHₐ—CHₑ—CH_{f})—(C₆H₅)₂]₃]₂); 8.29 (d, [Cu[Cₐ—C_{b}—COOH(CH_{c}—CHₐ—CHₑ—CH_{f})—(C₆H₅)₂]₃]₂]); 7.55 (m, [Cu[Cₐ—C_{b}—COOH(CH_{c}—CH_{d}—CHₑ—CH_{f})—(C₆H₅)₂]₃]₂]); 7.25 (m, [Cu[P[Ca-Cb-COOH(CHc-CHd-CHe-CHf)—(C₆H₅)₂]₃]₂]).

³¹P NMR (CDC₃): δ (ppm) 1.84 (s).

ESI-MS(+) in MeOH (m/z assignment, intensity %): 675 ([Cu(DPBA)₂]⁺, 100).

Compound CuL₂ BF₄ 11 L=P(DPMPP)₃

By following the procedure above reported compound Cu(P(DPMPP)₃)₂ BF₄ was obtained as a white solid.

MW 734

Anal. Calcd. for CuP₂C₃₈O₂H₃₄BF₄: C 62.10, H 4.66%. Found: C 61.45, H 4.62%. ESI(+)-MS (MeOH) (m/z assignment, intensity %): 551 ([Cu(DPMPP)₂]⁺, 100).

Compound CuL₂ BF₄ 12 L=P(POH)₃

By following the procedure above reported compound Cu(P(POH)₃)₂ BF₄ was obtained as a white solid.

MW 706

Anal. Calcd. for CuP₂C₃₆O₂H₃₀BF₄: C 61.16, H 4.28%. Found: C 60.35, H 4.22.

Compound CuL₂ BF₄ 13 L=P(TPPME)₃

By following the procedure above reported compound Cu(P(TPPME)₃)₂ BF₄ was obtained as a white solid.

MW 795

Anal. Calcd. for CuP₂C₄₀O₄H₃₈BF₄: C 60.43, H 4.82%. Found: C 58.11, H 4.81%.

Compound CuL₂ BF₄ 14 L=P(TPPMS)₃

By following the procedure above reported compound Cu(P(TPPMS)₃)₂ BF₄ was obtained as a white solid.

MW 835

Anal. Calcd. for CuP₂C₃₆O₆H₃₀BF₄: C 51.78, H 3.62%. Found: C 50.91, H 3.60.

### Example 3

### Evaluation of the Activity of the Compounds of the Invention

### Products and Methods

### Cell Cultures

Human breast (MCF-7), lung (H157), pancreatic (PSN-1) and colon (HCT-15 and LoVo) carcinoma cell lines were obtained from the American Type Culture Collection (ATCC, Rockville, MD). A431 are human cervical carcinoma cells kindly provided by Prof. F. Zunino (Molecular Pharmacology Unit, Experimental Oncology and Molecular Medicine, Istituto Nazionale dei Tumori, Milan, Italy). 2008 human ovarian cancer cells were kindly provided by Prof. G. Marverti (Dept. of Biomedical Science, University of Modena, Italy). The LoVo-OXP cells were derived using a standard protocol in which LoVo cells were grown in increasing concentrations of oxaliplatin and resistant clones were selected over a period of nine months (Gandin, V., et al., J. Cell. Mol. Med. 2012, 16, 142-151). Cell lines were maintained in the logarithmic phase at 37° C. in a 5% carbon dioxide atmosphere using the following culture media containing 10% fetal calf serum (Euroclone, Milan, Italy), antibiotics (50 units mL⁻¹ penicillin and 50 mg mL⁻¹ streptomycin), and 2 mM L-glutamine in: i) RPMI-1640 medium (Euroclone) for MCF-7, HCT-15, A431, 2008, PSN-1 and H157 cells; ii) F-12 HAM'S (Sigma Chemical Co.) for LoVo, LoVo MDR, and LoVo-OXP cells.

### Cytotoxicity Assay

The growth inhibitory effect towards tumor cell lines was evaluated by means of the MTT (3-(4,5-dimethylthiazol-2-yl)-2,5-diphenyltetrazolium bromide) assay (Alley, M. C., et al., Cancer Res. 1988, 48, 589-601). Briefly, depending upon the growth characteristics of the cell line, 3-8×10³ cells well⁻¹, were seeded in 96-well microplates in growth medium (100 μL) and then incubated at 37° C. in a 5% carbon dioxide atmosphere. After 24 h, the medium was removed and replaced with a fresh one containing the compound to be studied, at the appropriate concentration, dissolved in 0.9% sodium chloride solution just before use. Triplicate cultures were established for each treatment. After 72 h, each well was treated with 10 μL of a 5 mg mL⁻¹ MTT saline solution and, after 5 h of incubation, 100 μL of a sodium dodecylsulfate (SDS) solution in 0.01 M HCl were added. After an overnight incubation, the inhibition of cell growth induced by the tested complexes was determined by measuring the absorbance of each well at 570 nm using a Bio-Rad 680 microplate reader. Mean absorbance for each drug dose was expressed as percentage of the control and plotted vs. drug concentration. Dose-response curves were fitted and IC₅₀ values were calculated with four parameter logistic model (4PL). IC₅₀ values represent the drug concentrations that reduce the mean absorbance at 570 nm to 50% of those in the untreated control wells.

### Example 3a

### Cell Studies

Compounds 1-10 were evaluated for their cytotoxic activity towards a panel of human tumor cell lines including cervical (A431), colon (HCT-15), lung (H157), pancreatic (PSN-1), ovarian (2008) and breast (MCF-7) cancers as well as compounds 11-14 were tested on human ovarian cancer cells (2008). The cytotoxicity was evaluated by means of the MTT test for 72 h treatment with increasing concentrations of the tested compounds. For comparison purposes, the cytotoxicity of cisplatin, the most widely used anticancer metallo-drug, and oxaliplatin, key drug in FOLFOX (Folinic acid, 5-Fluorouracil & Oxaliplatin) regimens for the treatment of colorectal cancers, were evaluated in the same experimental conditions. IC₅₀ values (μM), calculated from dose-survival curves, are shown in Table 1.

TABLE 1
In vitro antitumor activity of compounds 1-10.

| | A431 | HCT-15 | H157 | PSN-1 | 2008 | MCF-7 |
|---|---|---|---|---|---|---|
| 1 | 0.2 ± 0.01 | 1.8 ± 0.8 | 1.6 ± 0.1 | 4.5 ± 1.8 | 2.2 ± 0.1 | 1.9 ± 0.7 |
| 2 | 3.0 ± 1.4 | 0.5 ± 0.2 | 3.4 ± 0.2 | 0.3 ± 0.08 | 1.3 ± 0.3 | 3.1 ± 0.6 |
| 3 | 3.1 ± 1.1 | 0.3 ± 0.2 | 1.2 ± 0.01 | 0.5 ± 0.2 | 0.5 ± 0.1 | 1.3 ± 0.4 |
| 4 | 0.8 ± 0.6 | 1.1 ± 0.5 | 0.9 ± 0.1 | 6.2 ± 0.9 | 1.9 ± 0.5 | 1.4 ± 0.3 |
| 5 | — | 8.0 ± 2.6 | 4.3 ± 1.2 | 7.3 ± 1.4 | 5.2 ± 1.7 | — |
| 6 | 7.7 ± 0.5 | 2.5 ± 0.1 | 3.8 ± 0.3 | 13.3 ± 3.2 | 14.5 ± 1.2 | 3.1 ± 0.9 |
| 7 | 15.0 ± 2.2 | 6.4 ± 0.5 | 8.4 ± 1.8 | 12.3 ± 1.5 | 9.2 ± 1.0 | 6.1 ± 1.5 |
| 8 | — | 10.2 ± 0.1 | 5.3 ± 1.2 | 7.7 ± 1.3 | 11.9 ± 0.1 | — |
| 9 | 4.7 ± 0.6 | 0.8 ± 0.2 | 1.5 ± 0.2 | 1.1 ± 0.5 | 0.6 ± 0.1 | 1.3 ± 0.3 |
| 10 | 3.8 ± 1.1 | 1.8 ± 0.6 | 0.9 ± 0.1 | 0.6 ± 0.01 | 0.3 ± 0.1 | 1.1 ± 0.2 |
| cisplatin | 2.1 ± 0.8 | 15.3 ± 2.6 | 2.1 ± 0.9 | 18.3 ± 3.1 | 2.2 ± 0.5 | 7.6 ± 0.2 |
| oxaliplatin | 3.7 ± 1.0 | 1.3 ± 0.3 | 11.8 ± 3.2 | 16.1 ± 2.5 | 1.7 ± 1.0 | 3.4 ± 1.2 |

Cytotoxic activity was examined against a panel of human tumor cell lines. For comparison purposes, the cytotoxicity of cisplatin and oxaliplatin were assessed under the same experimental conditions. The growth inhibitory effect was evaluated by means of MTT test. Cells (5-8×10³ mL⁻¹, depending on the growth characteristics of the cell line) were treated for 72 h with increasing concentrations of tested compounds. IC₅₀ values were calculated from the dose-survival curves by four parameter logistic model (P<0.05). SD=standard deviation, -=not detected. Compounds 1 and 4 were found, on average, 3.9 times more effective than cisplatin, and 3.1 time more effective than oxaliplatin. Similarly, compound 2 elicited an average cytotoxic activity 4 and 3.3 times higher than cisplatin and oxaliplatin, respectively. Compounds 3 and 10 were on average the most effective derivatives, showing an in vitro antitumor potential significantly higher than those of cisplatin (up to 6.8 times) and oxaliplatin (up to 5.5 times). Compound 9 proved to possess an average cytotoxic potency 4.8-and 3.8-fold higher compared to that of cisplatin and oxaliplatin, respectively. Compound 5 elicited average IC₅₀ values comparable to that of oxaliplatin but slightly lower than cisplatin, whereas compound 6 proved to be as effective as cisplatin but slightly less cytotoxic than oxaliplatin. Compounds 7 and 8 were found to possess, on average, a cytotoxic activity only slightly lower compared to the reference metal-based drugs.

Compounds 12-14 were tested on human ovarian cancer cells (2008). The cytotoxicity was evaluated by means of the MTT test for 72 h treatment with increasing concentrations of the tested compounds. For comparison purposes, the cytotoxicity of cisplatin and oxaliplatin were evaluated in the same experimental conditions. IC₅₀ values (μM), calculated from dose-survival curves, are shown in Table 2.

TABLE 2
In vitro antitumor activity of compounds 11-14.

| | 2008 |
|---|---|
| 11 | 0.4 ± 0.3 |
| 12 | 1.1 ± 0.3 |
| 13 | 1.5 ± 0.4 |
| 14 | 0.8 ± 0.2 |
| Cisplatin | 2.2 ± 0.5 |
| oxaliplatin | 1.7 ± 1.0 |

Cytotoxic activity was examined against a panel of human tumor cell lines. For comparison purposes, the cytotoxicity of cisplatin and oxaliplatin were assessed under the same experimental conditions. The growth inhibitory effect was evaluated by means of MTT test. Cells (5-8×10³ mL⁻¹, depending on the growth characteristics of the cell line) were treated for 72 h with increasing concentrations of tested compounds. IC₅₀ values were calculated from the dose-survival curves by four parameter logistic model (P<0.05). SD=standard deviation, -=not detected. Against human ovarian cancer cells, compounds 11-14 were much more effective than both cisplatin and oxaliplatin, with compound 11 being the most cytotoxic derivative.

Compounds 1-4, 6-7 and 9-10 have been also additionally tested for their in vitro antitumor activity in a pair of human cell lines which has been selected for their resistance to oxaliplatin (colon cancer cells LoVo/LoVo-OXP). A LoVo cell line retaining a multidrug resistance phenotype was also considered (LoVo MDR). Cross-resistance profiles were evaluated by means of the resistance factor (RF), which is defined as the ratio between the IC₅₀ value for the resistant cells and that arising from the sensitive cells (Table 3).

TABLE 3
Cross-resistance profiles

| | LoVo | LoVo-OXP | | LoVo MDR | |
|---|---|---|---|---|---|
| 1 | 0.4 ± 0.03 | 0.3 ± 0.1 | (0.8) | 0.5 ± 0.2 | (1.1) |
| 2 | 1.4 ± 0.1 | 1.3 ± 0.5 | (0.9) | 0.9 ± 0.6 | (0.6) |
| 3 | 0.5 ± 0.2 | 0.5 ± 0.1 | (0.9) | 0.9 ± 0.2 | (1.8) |
| 4 | 0.9 ± 0.3 | 0.7 ± 0.1 | (0.7) | 1.0 ± 0.1 | (1.1) |
| 6 | 0.7 ± 0.3 | 1.3 ± 0.5 | (1.8) | 1.2 ± 0.9 | (1.8) |
| 7 | 0.6 ± 0.3 | 0.4 ± 0.2 | (0.7) | 0.4 ± 0.3 | (0.7) |
| 9 | 0.6 ± 0.2 | 1.1 ± 0.6 | (1.7) | 0.8 ± 0.5 | (1.3) |
| 10 | 1.5 ± 0.4 | 0.09 ± 0.03 | (0.1) | 0.9 ± 0.4 | (0.6) |
| oxaliplatin | 3.1 ± 0.3 | 17.5 ± 1.8 | (17.0) | 1.4 ± 0.8 | (1.3) |

| | | | | |
|---|---|---|---|---|
| doxorubicin | 1.1 ± 0.5 | — | 19.4 ± 2.2 | (17) |

Cells (5×10³ mL-1) were treated for 72 h with increasing concentrations of tested compounds. Cytotoxicity was assessed by MTT test. IC₅₀ values (μM) were calculated by four parameter logistic model (p<0.05). -=not detected, S.D.=standard deviation. Resistant Factor (in brackets) is defined as IC₅₀ resistant/parent line.

The molecular mechanism involved in oxaliplatin resistance appeared to depend upon the decreased cellular accumulation (which is thought to be related to a greater activity of the ATP7B exporter rather than to the activity of P-glycoprotein (P-gp) and multi-drug resistance protein 1 (MRP1)) and a more efficient repair of oxaliplatin-induced DNA-damage by NER (Nucleotide Exci-sion Repair) (P. Noordhuis et al., Biochem. Pharmacol., 2008, 76, 53-61). LoVo OXP cells (derived from LoVo cells grown in the presence of increased concentration of oxaliplatin) were about 17-fold more resistant to oxaliplatin than parental cells. The data reported in Table 3 clearly indicate that all compounds possess resistance factors much lower than that of oxaliplatin, thus confirming their ability to overcome the oxaliplatin-resistance.

In Table 3 are also reported the results obtained in a multidrug resistant (MDR) colon carcinoma subline, LoVo MDR, in which the resistance to doxorubicin, a drug belonging to the MDR spectrum, is associated with an overexpression of multi-specific drug transporters, such as the 170 kDa P-glycoprotein (P-gp) (Wersinger, C., et al., Amino Acids 2000, 19, 667-685). It is well known that acquired MDR, whereby cells become refractory to multiple drugs, poses most important challenge to the success of anticancer chemotherapy. All copper derivatives tested against this cell line showed a similar response as for the parental subline, thus suggesting that they are not P-gp substrates and clearly underlining their ability to overcome MDR occurrence.

### Example 3b

### In Vivo Anticancer Activity Toward Lewis Lung Carcinoma

All studies involving animal testing were carried out in accordance with the ethical guidelines for animal research adopted by the University of Padua, acknowledging the Italian regulation and European Directive 2010/63/UE as to the animal welfare and protection and the related codes of practice. The mice were purchased from Charles River, Italy, housed in steel cages under controlled environmental conditions (constant temperature, humidity, and 12 h dark/light cycle), and alimented with commercial standard feed and tap water ad libitum. Animals were observed daily, and body weight and food intake recorded. The Lewis lung carcinoma (LLC) cell line was purchased from ECACC, UK. The LLC cell line was maintained in D-MEM (Euroclone) supplemented with 10% heat-inactivated FBS (Euroclone), 10 mM L-glutamine, 100 U mL⁻¹ penicillin, and 100 μg mL⁻¹ streptomycin in a 5% CO₂ air incubator at 37° C. The Lewis lung carcinoma (LLC) was implanted i.m. as a 2×10⁶ cell inoculum into the right hind leg of 8-week old male and female C57BL mice (24±3 g body weight). After 24 h from tumor implantation, mice were randomly divided into five groups (8 animals per group, 10 controls) and treated with a daily i.p. injection of compound 6 (5 mg kg⁻¹ in 0.9% NaCl solution) and 9 (3 mg kg⁻¹ dissolved in a vehicle solution composed of 0.5% DMSO (v/v) and 99.5% of saline solution (v/v)), cisplatin (1.5 mg kg⁻¹ in 0.9% NaCl solution), or the vehicle solution (0.5% DMSO (v/v) and 99.5% of saline solution (v/v)) from day 9 after tumor inoculation (palpable tumor). Compounds 6 and 9 are better tolerated than cisplatin and could be administered also at a greater dose (5 and 3 mg kg⁻¹, respectively, which represents ⅓ of the MTD). At day 15, animals were sacrificed, the legs were amputated at the proximal end of the femur, and the inhibition of tumor growth was determined according to the difference in weight of the tumor-bearing leg and the healthy leg of the animals expressed as % referred to the control animals. Body weight was measured every two days and was taken as a parameter for systemic toxicity. All the values are the means±S.D. of not less than three measurements. Multiple comparisons were made by Tukey-Kramer test (** p<0.01; * p or °p<0.05).

### Example 3c

### In Vivo Anticancer Activity Toward Lewis Lung Carcinoma

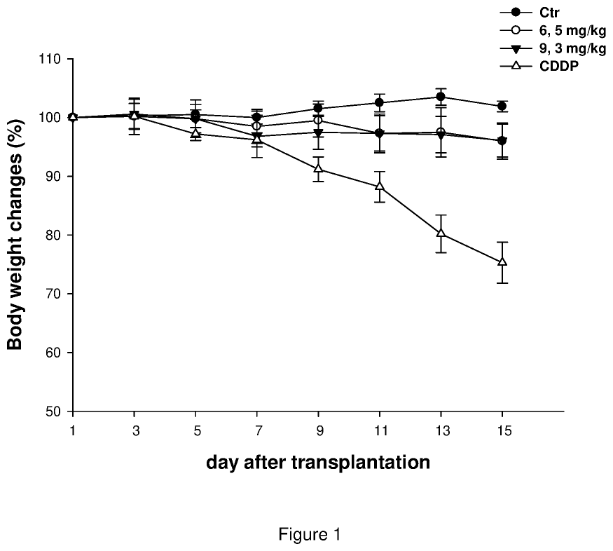

The in vivo antitumor activity of compounds 6 and 9 were evaluated in a model of solid tumor, the syngeneic murine Lewis lung carcinoma (LLC). Tumor growth inhibition induced by compounds 6 and 9 was compared with that promoted by the reference metallodrug cisplatin. From day 9 after tumor inoculation, when tumors became palpable, tumor-bearing mice received daily doses of compound 6 (5 mg kg⁻¹ in 0.9% NaCl solution) and 9 (3 mg kg⁻¹ dissolved in a vehicle solution composed of 0.5% DMSO (v/v) and 99.5% of saline solution (v/v)), cisplatin (1.5 mg kg⁻¹ in 0.9% NaCl solution), or the vehicle solution (0.5% DMSO (v/v) and 99.5% of saline solution (v/v)). Tumor growth was estimated at day 15, and the results are summarized in Table 4. For the assessment of the adverse side effects, changes in body weights of tumor-bearing mice were monitored daily (FIG. 1). The inhibition of tumor cell proliferation for the three compounds is shown in Table 4.

TABLE 4
In vivo anticancer activity toward
murine Lewis lung carcinoma (LLC).

| | Daily dose i.p. | Average tumor weight | Inhibition of |
|---|---|---|---|
| | (mg kg⁻¹) | (mean ± SD, g) | tumor growth (%) |
| Control^{a} | — | 0.589 ± 0.05 | — |
| 6 | 5 | 0.142 ± 0.09** | 75.9 |
| 9 | 3 | 0.098 ± 0.06** | 83.4 |
| cisplatin | 1.5 | 0.119 ± 0.10** | 79.8 |

^{a}Vehicle (0.5% DMSO (v/v) and 99.5% of saline solution (v/v)).

Starting from day 9 after tumor implantation, tested compounds were daily administered i.p. At day 15, mice were sacrified and tumor growth was detected as described above. Tukey-Kramer test ** p<0.01.

The tumor growth inhibition induced by compound 6 was compared to that promoted by the reference metallodrug cisplatin. Noteworthy, administration of compound 9 induced a 83% reduction of the tumor mass. Remarkably, the time course of body weight changes indicated that treatment with 6 and 9 did not induce significant body weight loss (<20%, FIG. 1). On the contrary, the time course of body weight changes indicated that cisplatin induces anorexia, with a body weight loss>20%.

1. A method for the treatment of tumours comprising the step of administering to a subject in need thereof a Cu(I) complex of Formula (I) 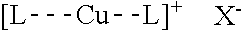
Wherein L is a ligand of Formula (II) 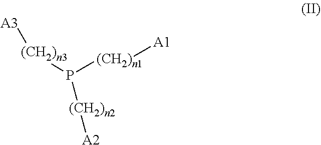
wherein
X is a monovalent anion
n1, n2, n3 are independently to each other, an integer from 0 to 1
A1, A2 and A3 are independently from each other,
a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
an heterocyclic ring selected from piperazinyl, morpholynyl, thiomorpholynyl, optionally substituted with (C₁-C₃)alkyl, where the heterocyclic ring is linked with the nitrogen atom to —CH₂— residue,
with the proviso that
when A1, A2 or A3 is an optionally substituted phenyl, then n1, n2 or n3, respectively, is 0, and
when A1, A2 or A3 is optionally substituted heterocyclic ring, then n1, n2 or n3, respectively, is 1.

2. The method according to claim 1, wherein A1, A2, A3 are, independently from each other, phenyl optionally substituted with a substituent selected from methyl, methoxy, F, formyl, sulphonyl hydroxyl, hydroxyl, methoxymethoxy and carboxyl, wherein at least one of A1, A2 and A3 is phenyl substituted with a substituent selected from methyl, methoxy, F, formyl, sulphonyl hydroxyl, hydroxyl, methoxymethoxy and carboxyl.

3. The method according to claim 2, wherein at least one of of A1, A2, A3, is phenyl substituted with methyl, F, or methoxy in para position.

4. The method according to claim 2, wherein at least one of of A1, A2, A3, is, phenyl substituted with formyl, sulphonyl hydroxyl, hydroxyl, methoxymethoxy or carboxyl in ortho position.

5. The method according to claim 1, wherein A1, A2, A3 are, independently from each other, an heterocyclic ring selected from piperazinyl, morpholynyl, thiomorpholynyl, optionally substituted with (C₁-C₃)alkyl, where the heterocyclic ring is linked with the nitrogen atom to —CH₂— residue.

6. The method according to claim 5, A1, A2, A3 are, independently from each other, substituted with methyl or ethyl.

7. The method according to claim 5, A1, A2, A3 are, independently from each other, piperazinyl, preferably substituted with methyl or ethyl.

8. The method according to claim 5, A1, A2, A3 are, independently from each other, morpholinyl or thiomorpholynyl, preferably unsubstituted.

9. The according to claim 1, wherein A1, A2, A3 are equal.

10. The method according to claim 1, wherein L comprises at least two of A1, A2, A3 equal, preferably optionally substituted, phenyl.

11. The method according to claim 1, wherein the legand L is selected from the group consisting of

| L | L |
|---|---|
| Formula (II) | Name |
| 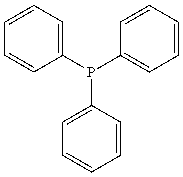 | PPh₃ 1 |
| 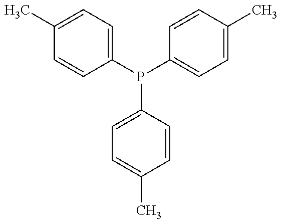 | P(p-tolyl)l₃ 2 |
| 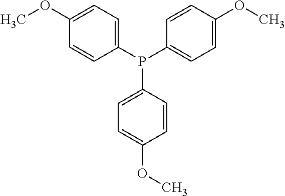 | P(p-OMe—C₆H₄)₃ 3 |
| 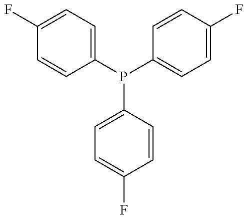 | P(p-F—C₆H₄)₃ 4 |
| 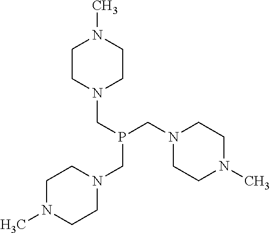 | PMepip 5 |
| 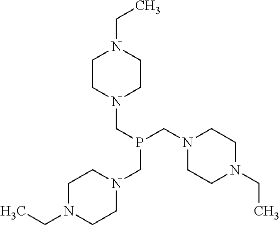 | PEtpip 6 |
| 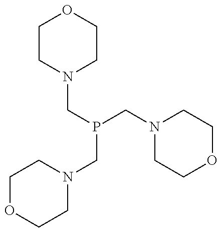 | Pmorf 7 |
| 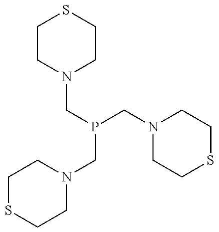 | Pthiomorf 8 |
| 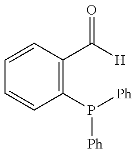 | DPBAL 9 |
| 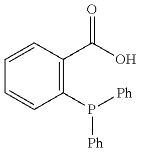 | DPBA 10 |
| 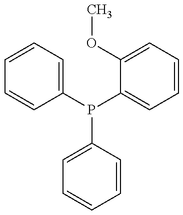 | DPMPP 11 |
| 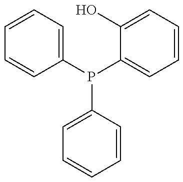 | POH 12 |
| 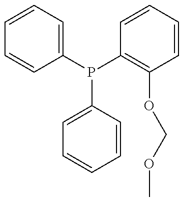 | TPPME 13 |
| 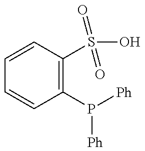 | TPPMS 14 |

12. The according to claim 1, wherein the Cu(I) is selected from the group consisting of

| Compound | L | Formula (II) |
|---|---|---|
| CuL₂NO₃ | PPh₃ Bis[tris(phenylphosphine)] copper(I) nitrate 1 | 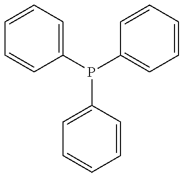 [SMILES: c1ccc(P(c2ccccc2)c2ccccc2)cc1] |
| CuL₂NO₃ | P(p-tolyl)l₃ Bis[tris(4- methylphenyl)phosphine] copper(I) nitrate 2 | 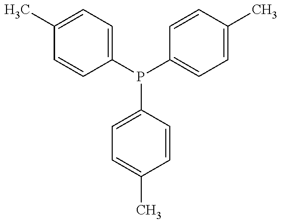 |
| CuL₂NO₃ | P(p-OMe—C₆H₄)₃ Bis[tris(4-methoxyphenyl) phosphine]copper(I) nitrate 4 | 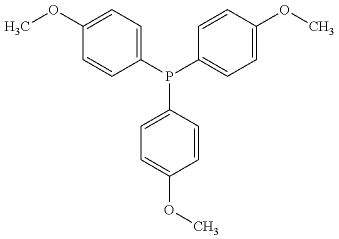 |
| CuL₂NO₃ | P(p-F—C₆H₄)₃ Bis[tris(4- fluorophenyl)phosphine] copper(I) nitrate 4 | 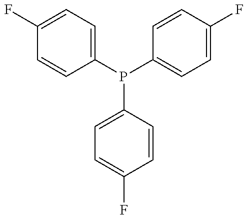 [SMILES: Fc1ccc(P(c2ccc(F)cc2)c2ccc(F)cc2)cc1] |
| CuL₂BF₄ | PMepip Bis[tris(N- methylpiperazineN′- methyl)phosphine] copper(I) tetrafluoroborate 5 | 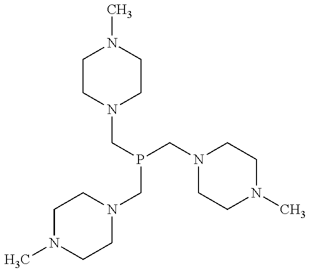 |
| CuL₂BF₄ | PEtpip Bis[tris(N- EthylpiperazineN′- methyl)phosphine] copper(I) tetrafluoroborate 6 | 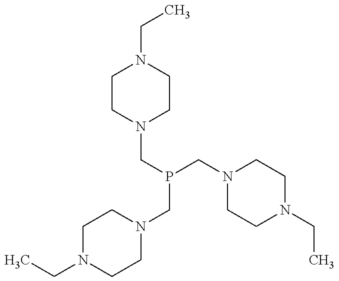 [SMILES: CCN1CCN(CP(CN2CCN(CC)CC2)CN2CCN(CC)CC2)CC1] |
| CuL₂BF₄ | Pmorf Bis[tris(morpholineN- methyl)phosphine] copper(I) tetrafluoroborate 7 | 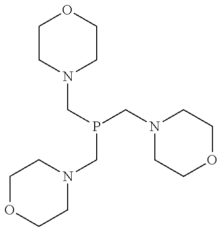 [SMILES: C1CN(CP(CN2CCOCC2)CN2CCOCC2)CCO1] |
| CuL₂BF₄ | Pthiomorf Bis[tris(thiomorpholineN- methyl)phosphine] copper(I) tetrafluoroborate 8 | 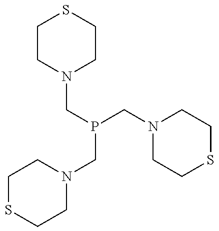 [SMILES: C1CN(CP(CN2CCSCC2)CN2CCSCC2)CCS1] |
| CuL₂BF₄ | DPBAL Bis[2- (diphenylphosphanyl) benzaldehyde] copper(I) tetrafluoroborate 9 | 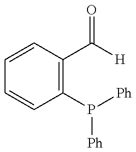 [SMILES: O=Cc1ccccc1P(c1ccccc1)c1ccccc1] |
| CuL₂BF₄ | DPBA Bis[2- (diphenylphosphanyl) benzoic acid] copper(I)tetrafluoroborate 10 | 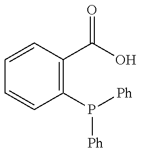 [SMILES: O=C(O)c1ccccc1P(c1ccccc1)c1ccccc1] |
| CuL₂BF₄ | DPMPP Bis[diphenyl(2- methoxyphenyl)phosphine] copper(I)tetrafluoroborate 11 | 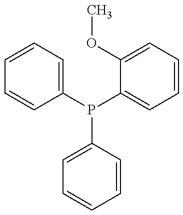 [SMILES: COc1ccccc1P(c1ccccc1)c1ccccc1] |
| CuL₂BF₄ | POH Bis[diphenyl(2- hydroxyphenyl)phosphine] copper(I)tetrafluoroborate 12 | 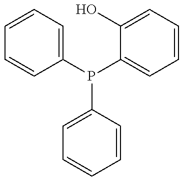 [SMILES: Oc1ccccc1P(c1ccccc1)c1ccccc1] |
| CuL₂BF₄ | TPPME Bis[diphenyl(2- (methoxymethoxy)phenyl) phosphine] copper(I)tetrafluoroborate 13 | 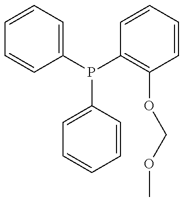 [SMILES: COCOc1ccccc1P(c1ccccc1)c1ccccc1] |
| CuL₂BF₄ | TPPMS Bis [2- (diphenylphosphino) benzenesulfonic acid] copper(I)tetrafluoroborate 14 | 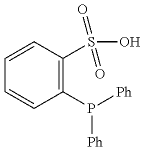 [SMILES: O=S(=O)(O)c1ccccc1P(c1ccccc1)c1ccccc1] |

13. A pharmaceutical composition comprising a Cu(I) complex of Formula (I) 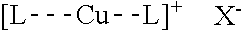
wherein L is a ligand of Formula (II) 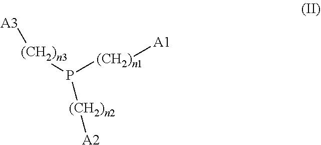
wherein
X⁻ is a monovalent anion
n1, n2, n3 are independently to each other, an integer from 0 to 1
A1, A2 and A3 are independently from each other,
a phenyl optionally substituted with (C₁-C₃)alkoxy, (C₁-C₃)alkyl, F, formyl, carboxyl, sulphonyl hydroxyl, hydroxyl, methoxy(C₁-C₃)alkoxy or
an heterocyclic ring selected from piperazinyl, morpholynyl, thiomorpholynyl, optionally substituted with (C₁-C₃)alkyl, where the heterocyclic ring is linked with the nitrogen atom to —CH₂— with the proviso that when A1, A2 or A3 is optionally substituted phenyl, then n1, n2 or n3, respectively, is 0, and
when A1, A2 or A3 is optionally substituted heterocyclic ring, then n1, n2 or n3, respectively, is 1, and pharmaceutically acceptable carrier.

14. A Cu(I) complex of Formula (I) 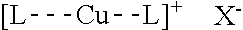
wherein L is a ligand of Formula (II) 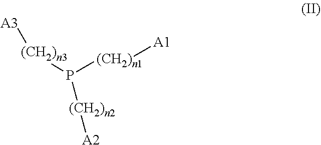
wherein
X⁻ is a monovalent anion
n1, n2, n3 are independently to each other, an integer from 0 to 1
A1, A2 and A3 are independently from each other,
a phenyl optionally substituted with a substituent selected from the group consisting of carboxyl, sulphonyl hydroxyl, hydroxyl, and methoxy(C₁-C₃)alkoxy or
an heterocyclic ring selected from piperazinyl, morpholynyl, thiomorpholynyl, optionally substituted with (C₁-C₃)alkyl, where the heterocyclic ring is linked with the nitrogen atom to —CH₂— residue,
with the proviso that
when A1, A2 or A3 is optionally substituted phenyl, then n1, n2 or n3, respectively, is 0, and at least one of A1, A2 and A3 is a phenyl substituted with a substituent selected from the group consisting of carboxyl, sulphonyl hydroxyl, hydroxyl, and methoxy(C₁-C₃)alkoxy.
when A1, A2 or A3 is optionally substituted heterocyclic ring, then n1, n2 or n3, respectively, is 1.

15. The Cu(I) complex according to claim 14, wherein A1, A2, A3 are, independently from each other, a phenyl optionally substituted with F, sulphonyl hydroxyl, hydroxyl, methoxymethoxy or carboxyl and at least one of A1, A2 and A3 is a phenyl substituted with sulphonyl hydroxyl, hydroxyl, methoxymethoxy or carboxyl.

16. The Cu(I) complex according to claim 14, wherein A1, A2, A3 are heterocyclic ring selected from piperazinyl, morpholynyl, thiomorpholynyl, optionally substituted with (C₁-C₃)alkyl, where the heterocyclic ring is linked with the nitrogen atom atom to —CH₂— residue.

17. The Cu(I) complex according to claim 16, wherein A1, A2, A3 are independently from each other heterocyclic ring substituted with methyl or ethyl.

18. The Cu(I) complex according to claim 17, wherein A1, A2, A3 are, independently from each other, piperazinyl, preferably substituted with methyl or ethyl.

19. The Cu(I) complex according to claim 16, A1, A2, A3 are, independently from each other morpholinyl or thiomorpholynyl, preferably unsubstituted.

20. The Cu(I) complex according to claim 14, wherein A1, A2, A3 are equal.

21. The Cu(I) complex according to claim 14, wherein L comprises at least two of A1, A2, A3 equal, preferably optionally substituted phenyl.

22. The Cu(I) complex according to claim 14, wherein the legand L is selected from the group consisting of

| L | |
|---|---|
| Formula (II) | L |
| 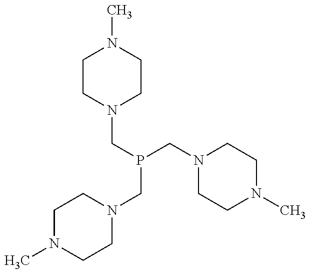 | PMepip 5 |
| 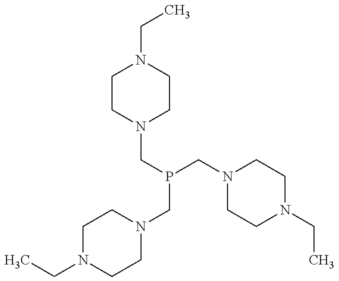 | PEtpip 6 |
| 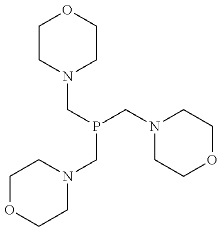 | Pmorf 7 |
| 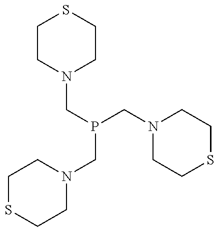 | Pthiomorf 8 |
| 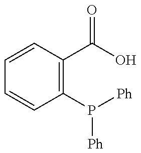 | DPBA 10 |
| 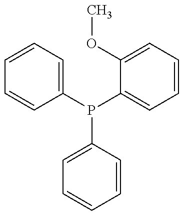 | DPMPP 11 |
| 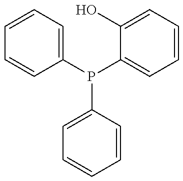 | POH 12 |
| 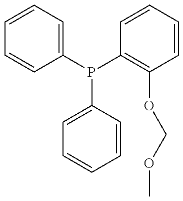 | TPPME 13 |
| 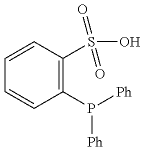 | TPPMS 14 |

23. The Cu(I) complex according to claim 14, wherein the Cu(I) complex is selected from the group consisting of

| Compound | L | Formula (II) |
|---|---|---|
| CuL₂BF₄ | PMepip 5 | 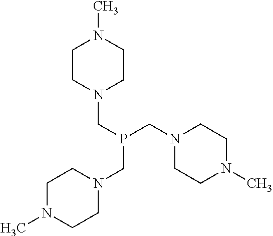 |
| CuL₂BF₄ | PEtpip 6 | 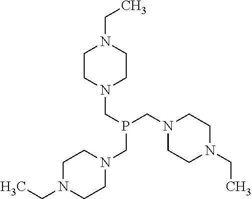 [SMILES: CCN1CCN(CP(CN2CCN(CC)CC2)CN2CCN(CC)CC2)CC1] |
| CuL₂BF₄ | Pmorf 7 | 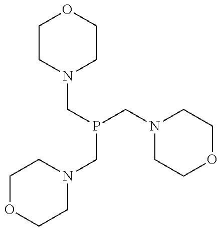 [SMILES: C1CN(CP(CN2CCOCC2)CN2CCOCC2)CCO1] |
| CuL₂BF₄ | Pthiomorf 8 | 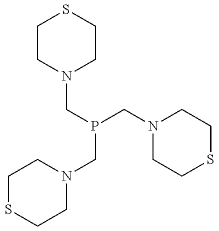 [SMILES: C1CN(CP(CN2CCSCC2)CN2CCSCC2)CCS1] |
| CuL₂BF₄ | DPBA 10 | 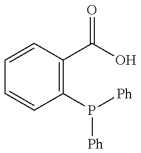 [SMILES: O=C(O)c1ccccc1P(c1ccccc1)c1ccccc1] |
| CuL₂BF₄ | DPMPP 11 | 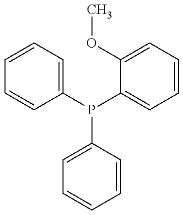 [SMILES: COc1ccccc1P(c1ccccc1)c1ccccc1] |
| CuL₂BF₄ | POH 12 | 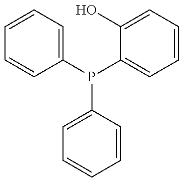 [SMILES: Oc1ccccc1P(c1ccccc1)c1ccccc1] |
| CuL₂BF₄ | TPPME 13 | 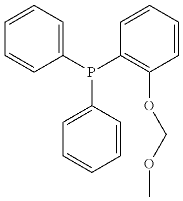 [SMILES: COCOc1ccccc1P(c1ccccc1)c1ccccc1] |
| CuL₂BF₄ | TPPMS 14 | 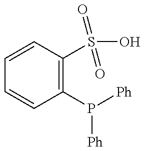 [SMILES: O=S(=O)(O)c1ccccc1P(c1ccccc1)c1ccccc1] |
